(19) United States
(12) Patent Application Publication
(10) Pub. No.: US 2024/0123178 A1
(43) Pub. Date: Apr. 18, 2024

(54) GAS-TREATMENT DEVICES

(71) Applicant: SMITHS MEDICAL INTERNATIONAL LIMITED, Ashford (GB)

(72) Inventors: Usamah Hanif, Southend-on-Sea (GB); Steven Mark Tupper, Hythe (GB); Christopher John Woosnam, London (GB); Timothy Bateman, Hythe (GB)

(73) Assignee: SMITHS MEDICAL INTERNATIONAL LIMITED, Ashford (GB)

(21) Appl. No.: 18/277,405

(22) PCT Filed: Feb. 28, 2022
(86) PCT No.: PCT/GB2022/000029

§ 371 Date: Aug. 16, 2023

(30) Foreign Application Priority Data
Mar. 15, 2021 (GB) 2103529.0

(51) Int. Cl. A61M 16/10; A61M 16/08

(52) U.S. Cl. CPC A61M 16/1045; A61M 16/0816; A61M 16/106

(57) ABSTRACT

An HME device has a housing (10) containing an HME element (25). The device has a first gas-flow passage through the device via the HME element (25) and a second gas-flow passage that by-passes the HME element. Flow is switched between the first or second flow passage by a pivoted shutter (30) that can be displaced by a rotatable knob (32).

This invention relates to gas-treatment devices of the kind including a gas-treatment element in a housing.

Where a patient breathes through a tube inserted in the trachea, such as a tracheostomy or endotracheal tube, gas flow to the bronchi is not warmed and moistened by passage through the nose. Unless the gas is warmed and moistened in some way it can cause damage and discomfort in the patient's throat. The gas can be conditioned by a humidifier in the ventilation circuit but, most conveniently, a heat and moisture exchange device (HME) is used. HMEs are small, lightweight devices including one or more exchange elements, such as of a paper or foam treated with a hygroscopic substance. When the patient exhales, gas passes through the exchange element and gives up a major part of its heat and moisture to the element. When the patient inhales, gas passes through the exchange element in the opposite direction and takes up a major part of the heat and moisture in the exchange element so that the gas inhaled by the patient is warmed and moistened. These HMEs are low cost and disposable after a single use. They can be connected in a breathing circuit or simply connected to the machine end of a tracheal tube and left open to atmosphere where the patient is breathing spontaneously. HMEs can be used with other breathing devices such as face masks.

HMEs are sold by Smiths Medical International Limited of Ashford, Kent, England under the Thermovent name (Thermovent is a registered trade mark of Smiths Medical International Limited), by Hudson RCI AB under the TrachVent name (TrachVent is a registered trade mark of Hudson RCI AB), by DAR, Medisize, Intersurgical and other manufacturers. Examples of HMEs are described in GB2391816, WO01/72365, U.S. Pat. No. 5,505,768, SE516666, U.S. Pat. No. 3,881,482, DE20302580, DE20114355U, WO97/01366, US2002/0157667, U.S. Pat. No. 6,422,235, EP1208866, U.S. Pat. No. 4,971,054, EP1699515, U.S. Pat. No. 5,035,236, EP535016, U.S. Pat. No. 5,647,344, GB2267840, EP856327, EP1699515, U.S. Pat. No. 7,363,925, WO15/107320, US2008/0099013, GB2540456 and WO2017/216508. The “Thermovent T” HME sold by Smiths Medical has a T-shape configuration with two HME elements mounted at opposite ends of a straight tubular housing extending transversely of the connection port by which the device is fitted onto a tracheostomy tube or the like. The tubular housing for the HME elements may be curved to follow the anatomical profile of the neck, as described in EP1888157.

HMEs are often used in breathing circuits for patients in intensive care where it may be necessary to be able to administer drugs by nebulisation into the lungs. It is not advisable to administer nebulised drugs via the HME media because this may interact or capture the nebulised drugs and reduce the amount reaching the lungs. It would also reduce the efficiency of the HME. In such circumstances the breathing circuit has to be disconnected to remove the HME and connect the nebuliser, leaving the patient without ventilation for a time. After the drugs have been administered the HME is reconnected in the breathing circuit, which, once again means that the patient is without ventilation for a time. This can be detrimental to the patient and takes time for the clinician. To avoid this a by-pass system could be used where an additional length of tubing is connected into the breathing system by a two-way valve and extends around the HME so that gas can flow through this tubing instead of through the HME when drugs need to be administered. These systems, however, tend to be bulky, cumbersome and add weight and dead space to the breathing circuit. The same problems exist with other gas-treatment devices, such as filters.

It is an object of the present invention to provide an alternative gas-treatment device.

According to the present invention there is provided a gas-treatment device of the above-specified kind, characterised in that the device has a first gas-flow passage through the device via the gas-treatment element, a second gas-flow passage through the device that by-passes the gas-treatment element, and a manually-displaceable member that in a first position blocks the second gas-flow passage and reveals the first gas-flow passage so that all gas flow passes through the gas-treatment element and in a second position blocks the first gas-flow passage and opens the second gas-flow passage so that all gas flow by-passes the gas-treatment element and passes through the second gas-flow passage.

The gas-treatment element is preferably an HME element or a filter. The manually-displaceable member may include a movable shutter, which in its first position blocks the second gas-flow passage and in its second position blocks the first gas-flow passage. The shutter preferably includes a planar member that is movable in the plane of the planar member. The shutter may be pivoted about an axis at right angles to the plane of the planar member. The shutter may include a manually-rotatable knob mounted with the planar member and aligned with the pivot axis so that the shutter can be moved between the first and second positions by rotating the knob. Alternatively, the manually-displaceable member may be a first part of the housing of the device including a gas inlet, the first part being rotatable relative to a second part of the housing between a first position in which the gas inlet on the first part forms a gas connection with the gas-treatment element and a second position in which the gas inlet on the first part forms a gas connection with the second gas-flow passage that by-passes the gas-treatment element.

An HME device according to the present invention will now be described, by way of example, with reference to the accompanying drawings, in which:

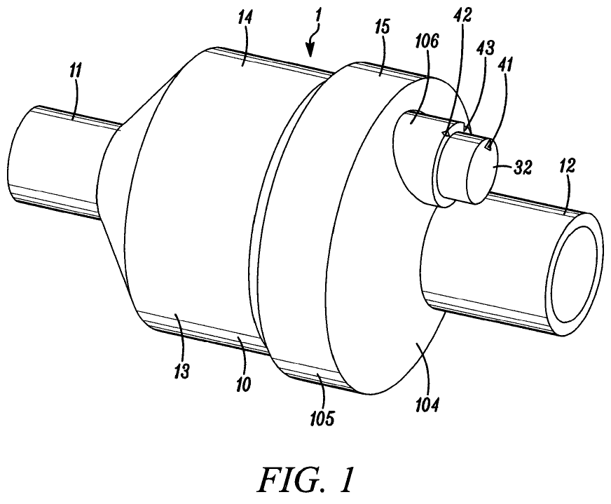

FIG. 1 is a perspective view of the HME device;

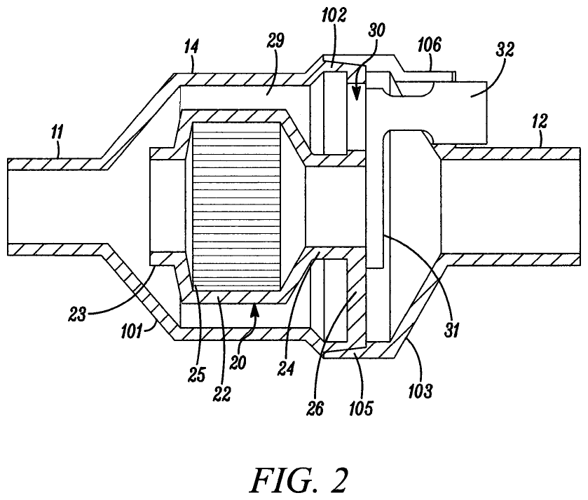

FIG. 2 is cross-sectional side elevation of the HME;

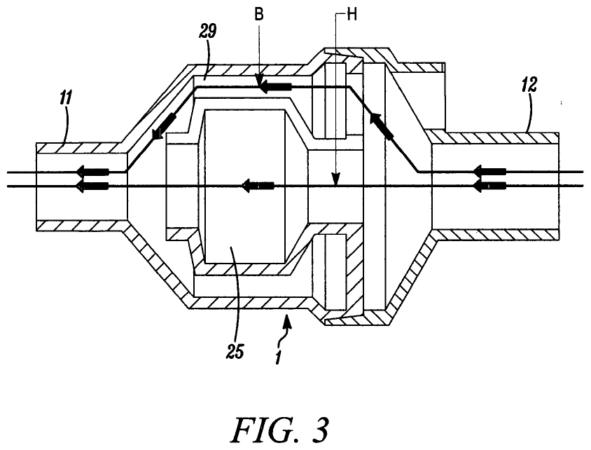

FIG. 3 is a cross-sectional side elevation view of the HME showing the alternative gas flow passageways through the device;

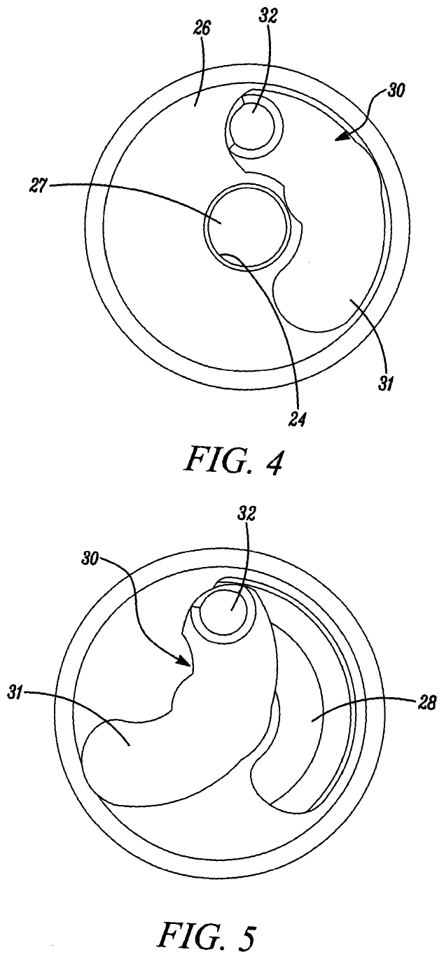

FIG. 4 is an end view from the machine end of the HME device with the machine end part of the housing removed and with device set to allow gas flow through the HME element;

FIG. 5 shows the same end view as in FIG. 4 but with the device set so that gas flow by-passes the HME element;

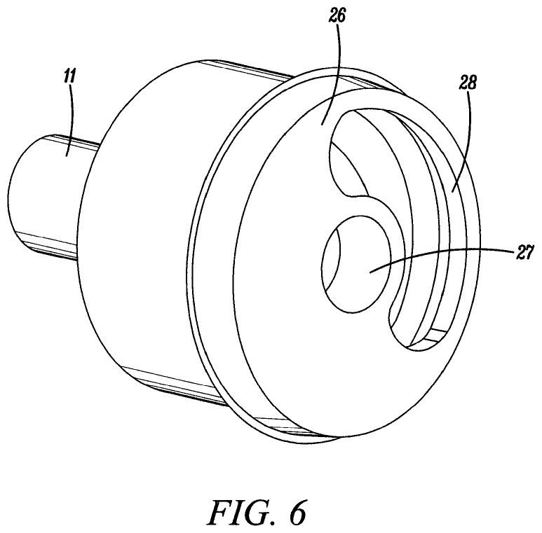

FIG. 6 is a perspective view of the machine end of the HME device with the machine end part of the housing and the manually-displaceable member removed;

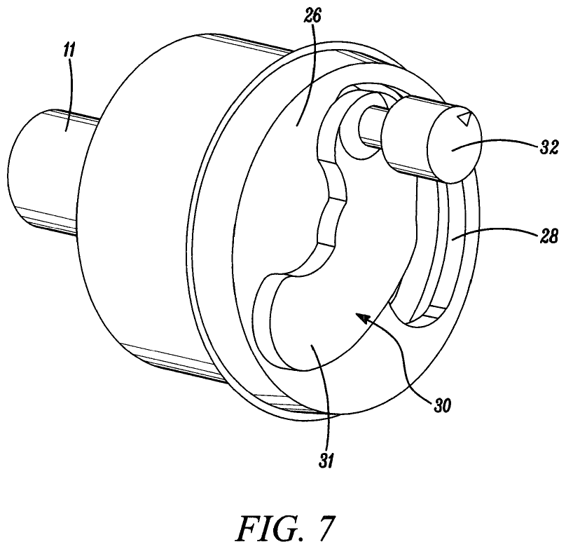

FIG. 7 is the same perspective view as in FIG. 6 but with the manually-displaceable member replaced and in a position where it causes gas flow to by-pass the HME element;

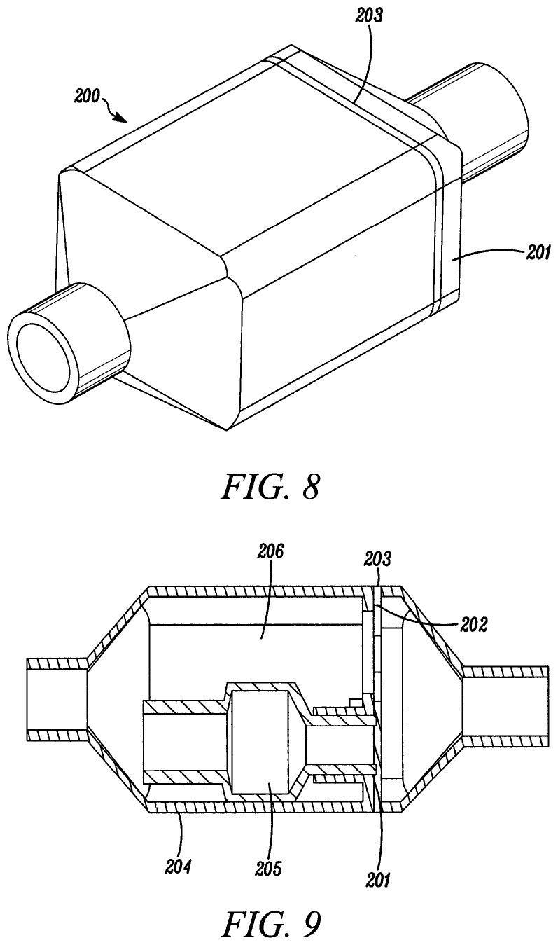

FIG. 8 is a perspective view of an alternative HME device;

FIG. 9 is a cross-sectional plan view of the device shown in FIG. 8;

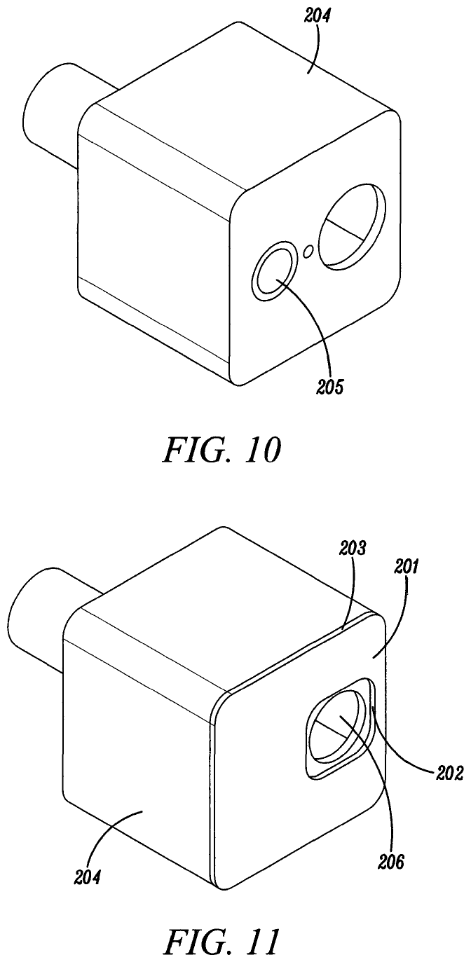

FIG. 10 is a perspective view of the machine end of the device of FIGS. 8 and 9 with the manually-displaceable member removed;

FIG. 11 is the same perspective view as in FIG. 10 but with the manually-displaceable member present and in the by-pass position; and

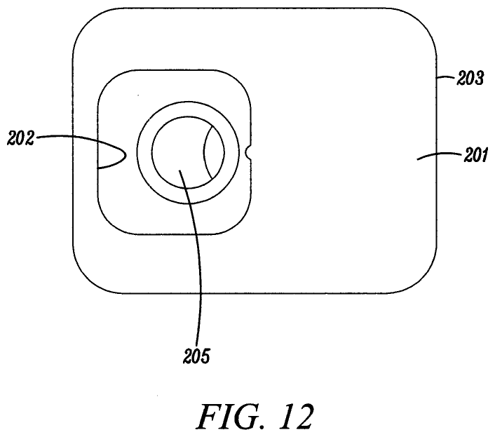

FIG. 12 is a view of the machine end of the device in FIGS. 8 and 9 with the machine end part of the housing removed and showing the device set in the HME mode.

With reference first to FIG. 1 to 3 there is shown an HME device 1 having an outer housing 10 of circular section with a patient end coupling or inlet 12 adapted to fit onto a mating male connector at the end of a breathing device (not shown) such as an endotracheal or tracheostomy tube. At the opposite end of the device 1 a machine end coupling or outlet 11 is axially aligned with the patient end coupling 12. Between the patient end coupling 12 and the machine end coupling 11 the housing 10 has a transversely or radially enlarged, stepped central region 13 having a major portion 14 towards the machine end and a shorter portion 15 of larger diameter towards the patient end. The housing 10 is formed of two parts. A machine end part 101 comprises the machine end coupling 11, the major portion 14 of the central region 13 and an inner ring 102 forming a part of the enlarged region 15. A patient end part 103 of the housing 10 comprises the patient end coupling 12, a flared or tapered end wall 104 and the outer part 105 of the enlarged region 15. The patient end part 103 of the housing 10 also has a short collar 106 projecting from the end wall 104 and projecting parallel to the axis of the device 1. The collar 106 supports a rotatable mode control knob 32, which will be explained in detail later.

The central region 13 of the outer housing 10 encloses an inner housing 20 of circular section arranged coaxially within the outer housing. The inner housing 20 has a central enlarged region 22 that tapers to a reduced diameter machine end 23 and patient end 24. The central enlarged region 22 contains a gas-treatment element in the form of an HME element 25 provided by a strip of corrugated paper treated with a hygroscopic salt to promote the absorption of moisture, the strip being wound into a circular coil. The corrugations of the paper strip are aligned parallel with the axis of the device so that air can flow along them. The inner housing 20 may be a separate component from the outer housing 10 secured with it in a conventional manner, such as by bonding, solvent, adhesive or welding. Alternatively, as shown in FIG. 3, the inner housing 20 is formed as a single, integral moulding with the outer housing 10, being supported within the outer housing by a radially-extending annular wall 26. As can be seen most clearly in FIG. 6, the wall 26 is solid apart from a central opening 27 through which the machine end 24 of the inner housing 20 opens. The wall 26 also has a slot 28 through it that is curved about a circular arc centred on the centre of the opening 27 and extending around about 120°. The outside of the inner housing 20 is spaced from the inside of the outer housing 10 to form an annular by-pass channel 29 around the outside of the inner housing. The slot 28 in the wall 26 opens into the by-pass channel 29 to enable gas communication between the machine and patient end couplings 11 and 12 via the by-pass channel 29.

The device 1 is completed by a manually-displaceable member 30 by which gas flow through the device can be selectively directed through either the HME element 25 (the arrowed path “H” in FIG. 3) or the by-pass channel 29 (via the arrowed path “B” in FIG. 3). As can be seen most clearly in FIG. 7, the manually-displaceable member takes the form of a shutter 30 having a curved plate 31 supported at one end by a circular knob 32 projecting at right angles from the plate. The plate 31 is the same shape as the slot 28 in the wall 26 but is slightly larger so that it overlaps the edges of the slot when it is aligned with it. The knob 32 is received in the collar 106 on the patient end part 103 of the outer housing 10 so that the shutter 30 is supported with one side of its plate 31 in slidable contact with the patient side surface of the wall 26. The knob 32 projects from the outer end of the collar 106 sufficiently to enable it to be gripped between finger and thumb. When the knob 32 is twisted to its full extent anti-clockwise the shutter plate 31 is similarly moved anti-clockwise until it lies over the curved slot 28 in the wall 26. This blocks passage of gas through the slot 28 and hence prevents flow along the by-pass channel 29. The shape of the shutter plate 31 is such that, in this position, the central opening 27 is open and revealed so that gas can flow freely along the central passage through the HME element 25. The knob 32 bears a visible marking 41 and the collar 106 bears two markings 42 and 43 respectively to indicate the extent of rotation of the knob. In the present example, with the shutter 30 in the HME mode, the marking 41 on the knob 32 is aligned with the left-hand marking 42 on the collar 106. When the clinician wishes to divert flow away from the HME 25 or other gas-treatment element he twists the knob 32 clockwise to its full extent so that the marking 41 on the knob aligns with the right-hand marking 43 on the collar 106. This swings the plate 31 clockwise away from the curved slot 28 until it lies over the central opening 27 and blocks gas flow through the HME element 25. Movement of the shutter plate 31 is limited by engagement with the inside of the outer housing 10. Friction between the shutter 30, the wall 26 and the housing 10 ensures that the plate 31 stays in the position to which it is displaced until it is moved to the other position by rotating the knob 32.

When the device 1 is set in the HME mode, as the patient exhales, warm, moist air flows forwardly through the patient coupling 12 and into the HME element 25 in one direction, from right to left in FIGS. 1 to 3. The warm, moist air flows through the HME element 25 along the spaces around the corrugations and between adjacent turns of the coil. As it does this the air gives up a large part of its heat and moisture to the HME element 25. The air then emerges through the central opening 27 in the wall 26 and flows forwardly out along the bore of the machine end coupling 11.

When the patient inhales, or when air is supplied by a ventilator or the like in the opposite direction, the air follows the same path through the device 1 but in the opposite direction. The inhaled air is cooler and drier than the exhaled air so, as this passes through the HME element 25, it takes up the major part of the warmth and moisture absorbed in the element during the previous exhalation phase thereby warming and moistening the air that flows to the patient.

The by-pass arrangement of the present invention avoids the need to disconnect the HME when administering nebulised medication in a compact device using a conventional HME element.

The HME element need not be made of a coil of corrugated, treated paper but could be of other materials used in conventional HME elements, such as foam or hollow fibres. It is not essential that the HME device and HME element be circular in section since they could be of other shapes such as oval or rectangular, although the circular shape has been found to give the highest efficiency. Although the HME described above has an axial form where the inlet and outlet are axially aligned other configurations are possible.

FIGS. 8 to 12 show an alternative HME device 200 where the manually-displaceable member that prevents or enables flow through the HME takes the form of a rectangular plate 201. The plate 201 has an offset opening 202 and is rotatably mounted about its centre about an axis “A” orthogonal to the plane of the plate and aligned with the axis of the HME device 200. The edge 203 of the plate 201 is exposed around the housing 204 so that it can be gripped and the plate rotated between two different positions. In one position (FIG. 12) the opening 202 aligns with a passage through the HME element 205 so that all flow to and from the patient is through the HME element. In the other position (FIGS. 9 and 11) the opening 202 is aligned with a by-pass channel 206 and the plate 201 blocks the passage to the HME element 205. The edge 203 of the plate 201 may have one or more surface formations (not shown) projecting slightly outwardly from the surface of the housing 204 to enable the plate to be gripped and rotated.

In other embodiments the manually-displaceable member need not be displaced by rotation but could, instead, for example, be displaced by a sliding, longitudinal displacement.

In an alternative embodiment a part of the outer housing itself could be displaceable to switch between the HME mode and the by-pass mode. For example, one end of the housing could have an off-centre coupling and be rotatably mounted on the main part of the housing. The HME element would be mounted in the housing off centre so that in one position of the rotatable end part of the housing its coupling would align with the HME element. In order to bypass the HME element during nebulisation or drug delivery the rotatable part of the housing would be twisted out of alignment with the HME element and into alignment with a bypass channel around the HME element.

The invention is not confined to HME devices but is applicable to other gas-treatment devices where it is desirable to be able to by-pass a gas-treatment element. For example, in a filter device it would be preferable for any nebulised or vapour substance to by-pass the filter element in order to avoid the filter capturing the substance and reducing the amount that flows to the lungs. Also, the effectiveness of a gas filter would be reduced by absorption of liquid substances.

1-7. (canceled)

8. A gas-treatment device including an gas-treatment element in a housing, characterised in that the device has a first gas-flow passage through the device via the gas-treatment element, a second gas-flow passage through the device that by-passes the gas-treatment element, and a manually-displaceable member that in a first position blocks the second gas-flow passage and reveals the first gas-flow passage so that all gas flow passes through the gas-treatment element and in a second position blocks the first gas-flow passage and opens the second gas-flow passage so that all gas flow by-passes the gas-treatment element and passes through the second gas-flow passage.

9. A gas-treatment device according to claim 8, characterised in that the gas-treatment element is an HME element or a filter.

10. A gas-treatment device according to claim 8, characterised in that the manually-displaceable member includes a movable shutter, which in its first position blocks the second gas-flow passage and in its second position blocks the first gas-flow passage.

11. A gas-treatment device according to claim 10, characterised in that the shutter includes a planar member that is movable in the plane of the planar member.

12. A gas-treatment device according to claim 11, characterised in that the shutter is pivoted about an axis at right angles to the plane of the planar member.

13. A gas-treatment device according to claim 11, characterised in that the shutter includes a manually-rotatable knob mounted with the planar member and aligned with the pivot axis so that the shutter can be moved between the first and second positions by rotating the knob.

14. A gas-treatment device according to claim 8, characterised in that the manually-displaceable member is a first part of the housing of the device including a gas inlet, and that the first part is rotatable relative to a second part of the housing between a first position in which the gas inlet on the first part forms a gas connection with the gas-treatment element and a second position in which the gas inlet on the first part forms a gas connection with the second gas-flow passage that by-passes the gas-treatment element.
